(19) United States
(12) Patent Application Publication
(10) Pub. No.: US 2003/0176765 A1
(43) Pub. Date: Sep. 18, 2003

(54) Devices for holding a body organ

(76) Inventor: Frederick J. Foley, Bedford, NH (US)

Correspondence Address: SHUMAKER & SIEFFERT, P. A., 8425 SEASONS PARKWAY, SUITE 105, ST. PAUL, MN, 55125, US

(21) Appl. No.: 10/348,210

(22) Filed: Jan. 17, 2003

Related U.S. Application Data
(60) Provisional application No. 60/351,539, filed on Jan. 23, 2002.

(51) Int. Cl. A61F 2/00

(52) U.S. Cl. 600/37

(57) ABSTRACT

In general, the invention provides devices and techniques for holding an organ, such as the apex of a beating heart. Some embodiments of the invention are directed to devices that include a manipulating device, a support shaft and a coupling mechanism that couples the manipulating device to the support shaft. In general, the coupling mechanism includes mating components. The mating components may be included in the manipulating device and/or the support shaft. When coupled, the mating components resist separation of the manipulating device and the support shaft. The mating components may also allow a degree of rotational freedom, and in some embodiments, may have a rotationally locked configuration and rotationally unlocked configuration.

This application claims priority from U.S. Provisional Application Serial No. 60/351,539, filed Jan. 23, 2002, the entire content of which is incorporated herein by reference.

### TECHNICAL FIELD

The invention relates to devices capable of providing adherence to organs of the body for purposes of medical diagnosis and treatment. More particularly, the invention relates to devices capable of adhering to, holding, moving, stabilizing or immobilizing an organ.

### BACKGROUND

In many areas of surgical practice, it may be desirable to manipulate an internal organ without causing damage to the organ. In some circumstances, the surgeon may wish to turn, lift or otherwise reorient the organ so that surgery or other therapy, such as thermal therapy, may be performed upon it. In other circumstances, the surgeon may simply want to move the organ out of the way. In still other cases, the surgeon may wish to hold the organ, or a portion of it, immobile so that it will not move during the surgical procedure. In further cases, it may be necessary to hold the organ being treated away from other organs or tissues. For example, when an organ is being treated with thermal therapy, in which heat may be applied to an organ for therapeutic purposes, the organ may be held away from other organs or tissues to prevent collateral injury.

Unfortunately, many organs are slippery and are difficult to manipulate. Holding an organ with the hands may be undesirable because of the slipperiness of the organ. Holding an organ may also be uncomfortable or hazardous to the surgeon when treating the organ with a therapy such as thermal therapy. Moreover, the surgeon's hands ordinarily cannot hold the organ and perform the procedure at the same time. The hands of an assistant may be bulky, becoming an obstacle to the surgeon. Also, manual support of an organ over an extended period of time can be difficult due to fatigue. Holding an organ with an instrument may damage the organ, especially if the organ is unduly squeezed, pinched or stretched. Holding an organ improperly may also adversely affect the functioning of the organ.

The heart is an organ that may be more effectively treated if it can be manipulated. Many forms of heart manipulation may be useful, including moving the heart within the chest and holding it in place. Some forms of heart disease, such as blockages of coronary vessels, may best be treated through procedures performed during open-heart surgery. During open-heart surgery, the patient is typically placed in the supine position. The surgeon performs a median sternotomy, incising and opening the patient's chest. Thereafter, the surgeon may employ a rib-spreader to spread the rib cage apart, and incise the pericardial sac to obtain access to the heart. For some forms of open-heart surgery, the patient is placed on cardiopulmonary bypass (CPB) and the patient's heart is arrested. Stopping the patient's heart is a frequently chosen procedure, as many coronary procedures are difficult to perform if the heart continues to beat. CPB entails trauma to the patient, with attendant side effects and risks. An alternative to CPB involves operating on the heart while the heart continues to beat. The surgeon may also choose to access the heart using a lateral thoracotomy with or without small portals to maintain an opening during the procedure.

Once the surgeon has access to the heart, it may be necessary to lift the heart from the chest or turn it to obtain access to a particular region of interest. Such manipulations are often difficult tasks. The heart is a slippery organ, and it is a challenging task to grip it with a gloved hand or an instrument without causing damage to the heart. Held improperly, the heart may suffer ischemia, hematoma or other trauma. The heart may also suffer a loss of hemodynamic function, and as a result may not pump blood properly or efficiently.

The problems associated with heart manipulation are greatly multiplied when the heart is beating. Beating causes translational motion of the heart in three dimensions. In addition, the ventricular contractions cause the heart to twist when beating. These motions of the heart make it difficult to lift the heart, move it and hold it in place.

In a coronary bypass operation, for example, the surgeon may need to manipulate the heart. The affected coronary artery may not be accessible without turning or lifting of the heart. Once the heart has been lifted or turned, the surgeon may need to secure the heart in a substantially fixed position.

### SUMMARY

In general, the invention provides devices and techniques for holding an organ. In a representative application, the invention is directed to devices and techniques for assembling an organ support apparatus that holds and supports the apex of a beating heart. As the heart beats, the heart bobs and twists. The twisting is problematic for at least two reasons. First, the twisting is important for the proper hemodynamic functioning of the heart, and therefore simply restraining the heart from all rotational motion has undesirable consequences upon hemodynamic functions. Second, the twisting compounds the difficulty of holding the heart with the manipulating device. The manipulating device may move and be difficult to control. Another potential difficulty is that the heart tissue may twist away from the manipulating device and may drop back into the chest or chafe against the manipulating device, which could result in heart trauma.

In some embodiments, the invention addresses these concerns by accommodating some degree of rotational freedom of the heart. An organ support system supports the heart, yet allows the heart a degree of freedom to rotate. In an exemplary embodiment of the invention, the heart is held by the apex with a vacuum-assisted manipulating device that includes a cup-like member and a skirt-like member. The manipulating device is supported by a support shaft such as a vacuum tube.

The invention is not limited to manipulation of the heart, nor is the invention limited to applications involving a vacuum-assisted manipulating device, nor is the invention limited to applications involving a manipulating device that is cup-shaped. On the contrary, the invention may be used to manipulate other organs, may be used with a manipulating device of any shape, and the manipulating device need not be vacuum-assisted. The invention may be implemented with a manipulating device that is irregularly shaped, for example, including projections that conform to the irregular shape of the organ. The invention may be implemented with a manipulating device that includes a plurality of vacuum-assisted appliances, or with a manipulating device that uses no vacuum pressure at all.

In one embodiment, the invention is directed to a device that includes a manipulating device that contacts an organ, a support shaft and a coupling mechanism that couples the manipulating device to the support shaft. In general, the coupling mechanism includes mating components. The mating components may be included in the manipulating device and/or the support shaft. When coupled, the mating components resist separation of the manipulating device and the support shaft. Mating components include, but are not limited to, flanges, apertures, pins, protrusions, sockets, grommets, threads, slots, liners, locking rings, recesses and various combinations thereof.

In some embodiments, the mating components are coupled with the assistance of a third mating component that is not included in either the manipulating device or the support shaft, such as a coupling ring. In other embodiments, the mating components included in the manipulating device and/or the support shaft are coupled directly to one another. In some embodiments, the coupling mechanism allows the manipulating device a degree of rotational freedom relative to the axis of the support shaft.

In another embodiment, the invention is directed to a method comprising engaging a manipulating device with an organ. The manipulating device is coupled to a support shaft with a coupling mechanism, and the coupling mechanism has a rotationally locked configuration and rotationally unlocked configuration. Vacuum pressure may be applied to cause the manipulating device to adhere to the organ. The method further includes placing the coupling mechanism in the rotationally locked configuration. This technique may be employed, for example, to hold the apex of a beating heart.

In a further embodiment, the invention is directed to a method comprising coupling a manipulating device to a support shaft with a coupling mechanism, engaging the manipulating device with an organ and substantially supporting the weight of the organ with the manipulating device. The method may further include placing the coupling mechanism in the rotationally locked configuration.

The invention can provide one or more advantages. For example, the invention may be applicable to many different kinds of manipulating devices and support shafts. Coupling mechanisms may also be of many different kinds, and may include features to support rotating, locking, rapid assembly or vacuum-assistance of the manipulating device. In some embodiments, coupling of the manipulating device to the support shafts with the coupling mechanism can be done in a matter of moments, with no special tools being required. Once the manipulating device is coupled to the support shaft, the manipulating device and the support shaft may cooperate to bear a load, such as the weight of a beating heart.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a perspective view of a manipulating device, a coupling mechanism and a support shaft in accordance with the invention, in conjunction with a beating heart.

FIG. 2A is a cross-sectional side view of a manipulating device, a support shaft and a coupling mechanism.

FIG. 2B is a plan view of the manipulating device, support shaft and coupling mechanism shown in FIG. 2A, with the coupling mechanism rotationally unlocked.

FIG. 2C is a plan view of the manipulating device, support shaft and coupling mechanism shown in FIGS. 2A and 2B, with the coupling mechanism rotationally locked.

FIG. 3A is a cross-sectional side view of another embodiment of a manipulating device, a support shaft and a coupling mechanism.

FIG. 3B is a plan view of the manipulating device, support shaft and coupling mechanism shown in FIG. 3A, with the coupling mechanism rotationally unlocked.

FIG. 4 is a cross-sectional side view of a further embodiment of a manipulating device, a support shaft and a coupling mechanism.

FIG. 5 is a cross-sectional side view of an additional embodiment of a manipulating device, a support shaft and a coupling mechanism.

FIG. 6 is a cross-sectional side view of another embodiment of a manipulating device, a support shaft and a coupling mechanism.

FIG. 7 is a cross-sectional side view of an alternative embodiment of a manipulating device, a support shaft and a coupling mechanism, the coupling mechanism including a fastening grommet and a locking ring.

### DETAILED DESCRIPTION

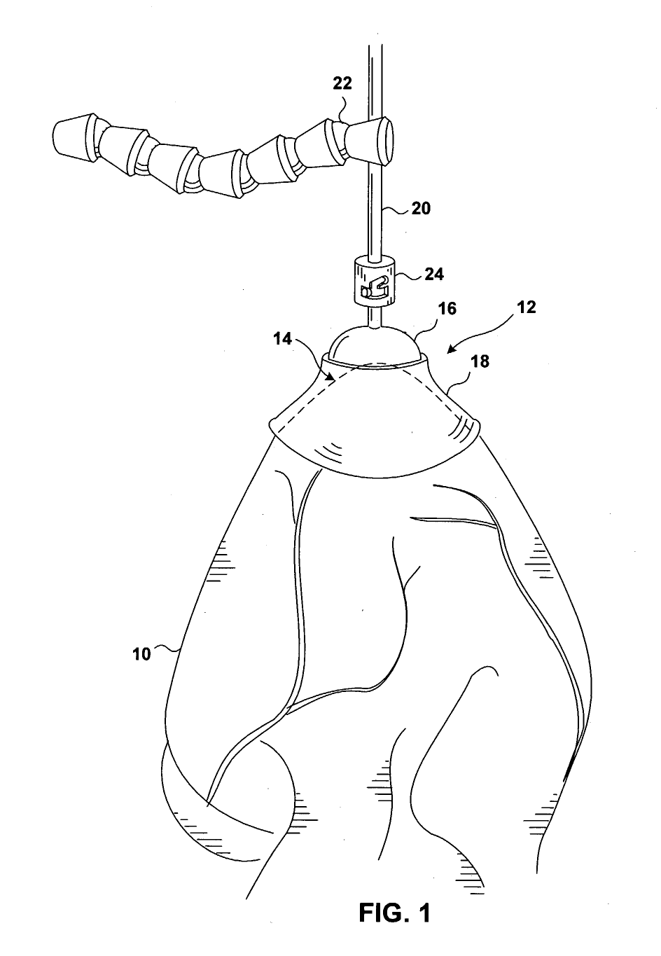

FIG. 1 is a perspective view of a heart 10, which is being held by a manipulating device 12. In the exemplary application shown in FIG. 1, a surgeon (not shown in FIG. 1) has obtained access to heart 10 and has placed manipulating device 12 over the apex 14 of heart 10. The surgeon has lifted apex 14 with manipulating device 12, giving the surgeon access to a desired region of heart 10. Although held by manipulating device 12, heart 10 has not been arrested and continues to beat. Beating causes heart 10 to move in three dimensions. In particular, heart 10 moves in translational fashion, by bobbing up and down and by moving from side to side. Heart 10 also expands and contracts as heart 10 fills with and expels blood. Heart 10 may twist as it expands and contracts.

Manipulating device 12 may engage heart 10 using any of a number of techniques. In FIG. 1, manipulating device 12 is an exemplary device that includes a cup-like member 16 and a skirt-like member 18 extending outward from cup-like member 16. Manipulating device 12 adheres to apex 14 with the aid of vacuum pressure supplied from a vacuum source (not shown in FIG. 1) via a vacuum tube 20. Vacuum tube 20 serves as a support shaft for manipulating device 12 and as a supply of vacuum pressure. Alternatively, manipulating device 12 may be supported by a dedicated support shaft, with vacuum tube 20 providing little or no support.

Upon application of vacuum pressure, skirt-like member 18 deforms and substantially forms a seal against the surface of the tissue of heart 10. Skirt-like member 18 is formed of a compliant material that allows the seal to be maintained even as heart 10 beats. Adherence between heart 10 and manipulating device 12 may be promoted by other factors as well, such as a tacky surface of skirt-like member 18 placed in contact with heart 10.

Manipulating device 12 illustrates the practice of the invention. The invention is not limited to manipulating device 12, however. The invention may be practiced with a manipulating device that is not vacuum-assisted, or a manipulating device that is not cup-shaped, or a manipulating device that lacks a skirt-like member. The invention may be practiced with manipulating devices that include multiple sites of organ contact, manipulating devices that have single-piece or multi-piece construction, and manipulating devices that include additional structural features such as a handle or a pressure valve. The invention may be practiced with manipulating devices of any shape.

Moreover, the invention may be practiced with support shafts of many types. The support shaft may be, for example, thick, thin, rigid, flexible, telescoping, articulating, hollow, solid, of a variety of shapes and made of a variety of materials.

The surgeon may move heart 10 by moving manipulating device 12 and/or vacuum tube 20. When the surgeon has obtained access to certain areas of heart 10, the surgeon may desire to maintain heart 10 in a substantially fixed position. In the exemplary application shown in FIG. 1, the surgeon suspends heart 10 by apex 14 and prepares to hold heart 10 in place with a securing structure 22. Securing structure 22 may include, for example, an adjustable support arm that can be locked in a variety of positions. The support arm may be affixed to a relatively immovable object, such as a rib spreader (not shown) or an operating table (not shown).

A coupling mechanism 24 couples manipulating device 12 to the support shaft or vacuum tube 20. As will be described below, coupling mechanism 24 includes a translational lock that can bear an applied load, such as the weight of heart 10. Although heart 10 may be held in tension by its own weight, coupling mechanism 24 permits some rotational motion. Accordingly, manipulating device 12 may rotate to a degree relative to vacuum tube 20. In some embodiments of the invention, coupling mechanism 24 includes a rotational lock that restricts rotational motion.

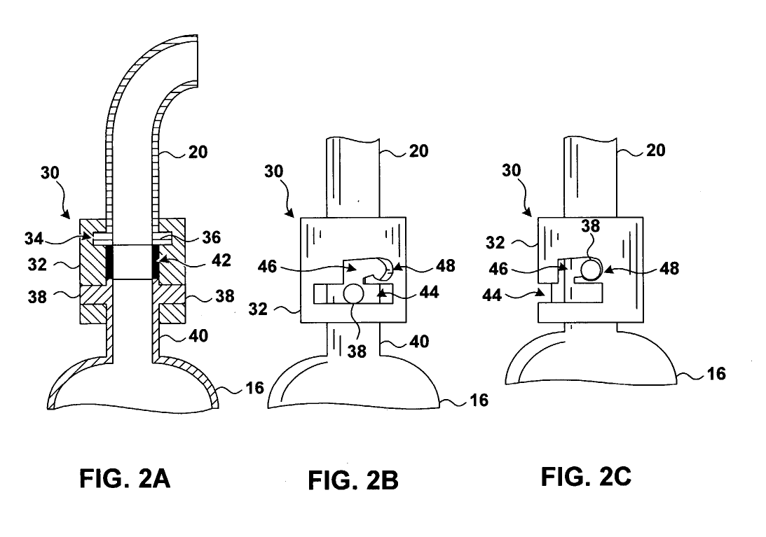

FIG. 2A is a cross-sectional side view of an exemplary coupling mechanism 30. Coupling mechanism 30 is shown in the exemplary application depicted in FIG. 1, but the exemplary application depicted in FIG. 1 is not limited to coupling mechanism 30.

Coupling mechanism 30 includes a coupling ring 32 that is separate from vacuum tube 20 and cup-like member 16. Coupling ring 32 may include a recess 34 that receives a retaining ring 36 in vacuum tube 20. Coupling ring 32 may also include slots, which will be shown more clearly in FIGS. 2B and 2C, that receive one or more pins 38. Pins 38 protrude from a shaft member 40 that extends proximally from cup-like member 16. Cup-like member 16, shaft member 40 and pins 38 may be integrally formed from a single material.

Coupling mechanism 30 may optionally include gasket material 42. Gasket material 42, which may be substantially more pliable than cup-like member 16 or vacuum tube 20, may serve many purposes. First, gasket material 42 helps provide a seal when vacuum tube 20 serves as the support shaft, thereby preventing loss of vacuum pressure. Second, gasket material 42 bears against cup-like member 16 and vacuum tube 20, to separate cup-like member 16 from vacuum tube 20. In other words, gasket material 42 may have a degree of elasticity, resulting in elastic force that biases cup-like member 16 and vacuum tube 20 to move apart from one another. Third, gasket material 42 may be compressed to allow coupling ring 32 to assume a rotationally locked configuration, and bears against cup-like member 16 and vacuum tube 20 to maintain the rotationally locked configuration. Gasket material 42 may be made of a pliable, biocompatible material such as silicone.

A rotationally unlocked configuration is shown in FIG. 2B. Pin 38 rides in horizontal slot 44. As cup-like member 16 rotates relative to the axis of vacuum tube 20, shaft member 40 and pin 38 also rotate, with pin 38 sliding in horizontal slot 44.

Coupling ring 32 also includes a locking slot 46, which extends perpendicularly from horizontal slot 44 and then substantially parallel to horizontal slot 44. Pin 38 is ordinarily prevented from entering locking slot 46 by gasket material 42, which separates cup-like member 16 from vacuum tube 20.

FIG. 2C shows coupling mechanism 30 in a rotationally locked configuration. The surgeon has pushed cup-like member 16 toward vacuum tube 20, compressing gasket material 42 and causing pin 38 to enter locking slot 46. By twisting coupling ring 32, the surgeon has slid pin 38 substantially horizontally in locking slot 46 and has seated pin 38 in recess 48. Once in recess 48, pin 38 is held in recess 48 by gasket material 42, and is prevented from rotating relative to the axis of vacuum tube 20. Accordingly, cup-like member 16 and shaft member 40 are prevented from rotating relative to the axis of vacuum tube 20.

Cup-like member 16, vacuum tube 20 and coupling mechanism 30 may be assembled by, for example, snapping the components together. Gasket material 42 may be coupled to shaft member 40, or vacuum tube 20, or both, prior to assembly.

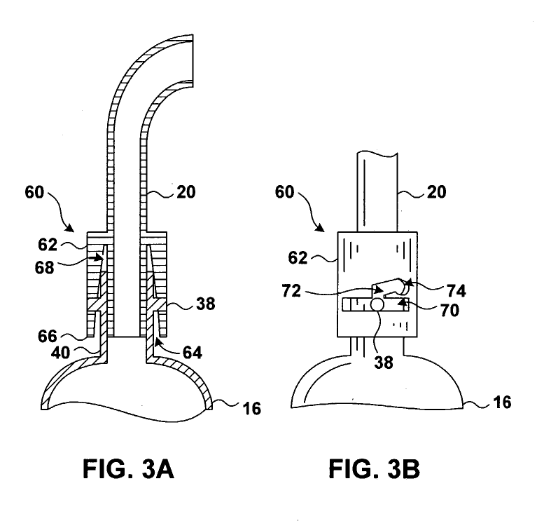

FIG. 3A is a cross-sectional side view of another exemplary coupling mechanism 60. Coupling mechanism 60 is similar to coupling mechanism 30 in that coupling mechanism 60 includes a coupling ring 62. Coupling ring 62, however, is not separate from vacuum tube 20, but may be integrally formed with vacuum tube 20.

Cup-like member 16 includes shaft member 40, from which one or more pins 38 protrude. Pins 38 are received by slots in coupling ring 62, as will be shown in FIG. 3B. Coupling mechanism 60 may be assembled by inserting the proximal end of cup-like member 16 into opening 64. Flanges 66 around opening 64 may deform to permit entry of pins 38. Pins 38 may then snap into a slot in coupling ring 62. Flanges 66 may include a tapered inner wall 68 that bears against the proximal end of cup-like member 16 and tends to push cup-like member 16 distally.

FIG. 3B shows coupling mechanism 60 in a rotationally unlocked configuration. Like coupling ring 32, coupling ring 62 includes a horizontal slot 70, a locking slot 72 and a recess 74. Coupling mechanism 30 may be placed in a rotationally locked configuration by pushing cup-like member 16 further into opening 64, causing pin 38 to enter locking slot 72, and twisting coupling ring 62 to seat pin 38 in recess 74. Once in recess 74, pin 38 is held in recess 74 by tapered inner wall 68 of flanges 66.

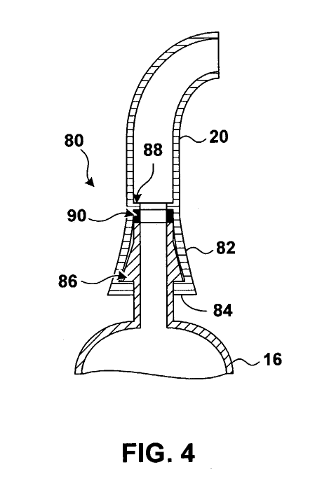

FIG. 4 is a cross-sectional side view of another exemplary coupling mechanism 80. In this embodiment, vacuum tube 20 includes a flared distal end 82 with a distal flange 84. Flared distal end 82 receives cup-like member 16, which includes a complementary flange 86. Coupling mechanism 80 is assembled by pushing the proximal end of cup-like member 16 into the opening defined by distal flange 84, until complementary flange 86 snaps inside vacuum tube 20. At this point, distal flange 84 engages complementary flange 86. Vacuum tube 20 may include a proximal flange 88, which prevents cup-like member 16 from moving too far proximally. Proximal flange 88 also bears against gasket material 90, which may seal leaks and hold cup-like member 16 in position. Gasket material 90, which may be made of a pliable, biocompatible material such as silicone, may be coupled to cup-like member 16, or vacuum tube 20, or both, prior to assembly. Alternatively, gasket material 90 may be omitted.

Once distal flange 84 engages complementary flange 86, distal flange 84 and complementary flange 86 may rotate relative to one another. The rotational freedom may be restricted by the friction between distal flange 84 and complementary flange 86. The embodiment shown in FIG. 4 does not show any mechanism for placing coupling mechanism 80 in a rotationally locked configuration. Such a mechanism may be included, however.

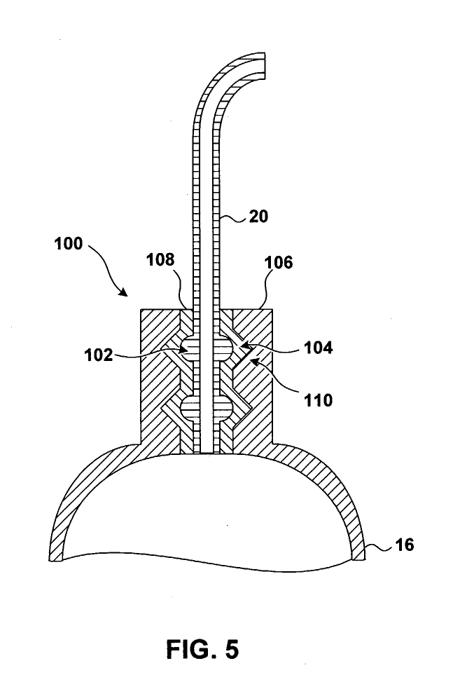

FIG. 5 is a cross-sectional side view of an additional exemplary coupling mechanism 100. Vacuum tube 20 includes one or more protrusions 102 that are received by complementary recesses 104 included in cup-like member 16. In this embodiment, cup-like member 16 may include a rigid receptacle 106 lined with a pliable liner 108. Pliable liner may be made of a pliable, biocompatible material such as silicone. When the distal end of vacuum tube 20 is inserted into receptacle 106, liner 108 deforms to allow protrusions 102 to enter. When protrusions 102 line up with recesses 110 in receptacle 106, protrusions 102 snap into recesses 110. In addition to holding protrusions 102 in recesses 110, liner 108 helps provide a seal that prevents a loss of vacuum pressure.

Once protrusions 102 are seated in recesses 110, the rotational freedom of vacuum tube 20 about its axis may be restricted by the friction. Alternatively, a locking mechanism (not shown in FIG. 5) may be used to place coupling mechanism 100 in a rotationally locked configuration.

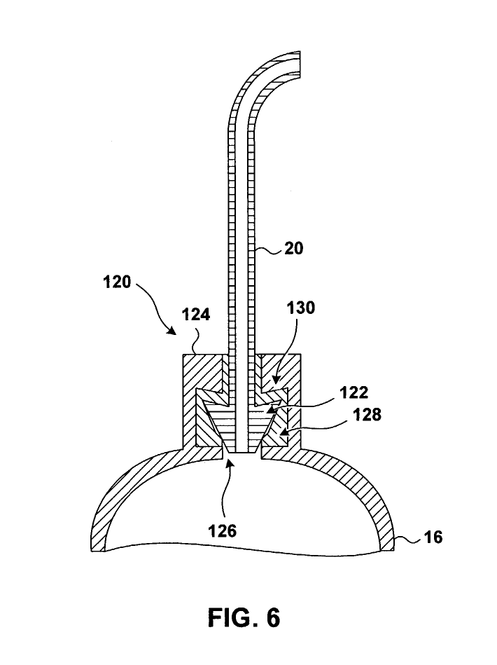

FIG. 6 is a cross-sectional side view of a further exemplary coupling mechanism 120. In this embodiment, vacuum tube 20 includes a tapered distal end 122 that mates with receptacle 124 of cup-like member 16. Receptacle 124 includes a socket 126 that receives tapered distal end 122.

Socket 126 may be lined with a pliable liner 128. When tapered distal end 122 of vacuum tube 20 is inserted into receptacle 124, liner 128 deforms to allow tapered end 122 to enter. Tapered end 122 may also deform upon entry into receptacle 124. When tapered end 122 is fully inserted in receptacle 124, tapered end expands in socket 126, and flanges 130 prevent tapered end 122 from being easily withdrawn from socket 126. Like liner 108 in coupling mechanism 100 shown in FIG. 5, liner 128 helps provide a seal that prevents a loss of vacuum pressure.

Once tapered end 122 is seated in socket 126, the rotational freedom of vacuum tube 20 about its axis may be restricted by the friction. A locking mechanism (not shown in FIG. 6) may be used to set coupling mechanism 120 in a rotationally locked configuration.

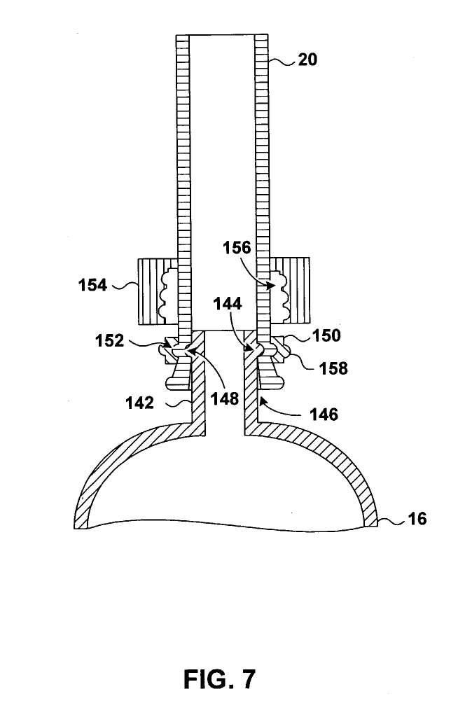

FIG. 7 is a cross-sectional side view of an additional exemplary coupling mechanism 140. Cup-like member 16 includes shaft member 142 that extends proximally from cup-like member 16 and includes male flange 144. When inserted in flared opening 146 of vacuum tube 20, vacuum tube 20 may deform to receive male flange 144. Male flange may seat in mating groove 148 in vacuum tube 20.

Male flange 144 may be, for example, an annular projection from shaft member 142. In another embodiment, male flange may be spirally wound around shaft member 142 like the thread of a screw. In similar fashion, mating groove 148 may spiral around the inner surface of vacuum tube 20. In this embodiment, shaft member 142 of cup-like member 16 may be twisted into flared opening 146.

In some circumstances, vacuum tube 20 is formed from a flexible material. In such a case, the engagement between shaft member 142 and vacuum tube 20 may not be very secure. Very little force may be needed to cause vacuum tube 20 to deform and for male flange 144 to slip from mating groove 148. Accordingly, a fastener such as grommet 150 may surround the interface between cup-like member 16 and vacuum tube 20. Grommet 150 may, for example, snap over or twist over male projections 152 on the exterior surface of vacuum tube 20. Grommet 150 may provide a more secure connection between shaft member 142 and vacuum tube 20. Grommet 150 may be sufficiently tight to prevent male flange 144 from slipping from mating groove 148, but sufficiently loose to accommodate some rotational motion.

A locking ring 154 may also be provided for additional security. Locking ring 154 may include internal threads 156 that engage with an external thread 158 on grommet 150. Internal threads 156 are tapered such that locking ring 154 squeezes grommet 150 more tightly the farther locking ring is screwed over grommet 150. As locking ring 154 squeezes grommet 150, grommet 150 squeezes vacuum tube 20, thereby increasing the frictional engagement between shaft member 142 and vacuum tube 20. When locking ring 154 is twisted tightly over grommet 150, cup-like member 16 may be effectively locked in position relative to vacuum tube 20.

There are many variations on the devices shown in FIG. 7. For example, shaft member 142 may include a mating groove on its exterior surface rather than male flange 144, and instead of mating groove 148, vacuum tube 20 has a mating flange. In addition, the device may be modified such that vacuum tube 20 is inserted into an opening in cup-like member 16, rather than the other way around. In some embodiments, grommet 150 may be optional, with locking ring 154 serving as the fastener and as the locking mechanism.

The invention can provide one or more advantages. Manipulating devices of infinite variety can be coupled to vacuum tubes or support shafts of infinite variety. The assembly is simple and takes only a few moments. In many cases, no special tools are needed for assembly. Once the manipulating device is coupled to the support shaft, the manipulating device and the support shaft may cooperate to bear a load, such as the weight of a beating heart.

Some embodiments allow considerable rotational motion at the site of coupling, some allow little rotational motion, and others allow no rotational motion at all. The surgeon may select a coupling mechanism that the surgeon feels is best for the patient's needs. Some coupling mechanisms give the surgeon the option of allowing rotational motion in one configuration, and being rotationally locked in another configuration. Even when the coupling mechanisms themselves provide limited or no rotational freedom, the coupling mechanisms generally do not foreclose rotational freedom from being provided by other means. For example, a flexible support shaft may allow a degree of twisting, and thus may provide some rotational freedom even if the coupling mechanism does not.

Various embodiments of the invention have been described. These embodiments are illustrative of the practice of the invention. Although the figures demonstrate implementations with a manipulating device that is vacuum-assisted and is substantially cup-shaped, the invention may be used with a manipulating device of any shape, and the manipulating device need not be vacuum-assisted. A manipulating device may be irregularly shaped, for example, including projections that extend radially outward from the center of the manipulating device and conform to the irregular shape of heart 10. In another context, the manipulating device may include a plurality of vacuum-assisted appliances, or a manipulating device may use no vacuum pressure at all.

In addition, the figures demonstrate implementations in which a vacuum tube is also the support shaft for the manipulating device. The invention is not limited to applications in which the vacuum tube is also the support shaft. The various embodiments may be adapted for use with a support shaft that lacks a lumen for conveying vacuum pressure. Indeed, some of the embodiments may be better suited for use with a solid support shaft than with a vacuum tube. Some embodiments work well with flexible support shafts and other embodiments work well with rigid support shafts. The invention encompasses all of these embodiments.

The embodiments described above also demonstrate an interchangeability of functions. A figure may show a flange associated with a manipulating device, for example, but the coupling mechanism may be easily reversed, such that the flange is associated with the support shaft or vacuum tube. In some embodiments, the manipulating device is inserted into the support shaft, and in other embodiments, the opposite is true. The invention encompasses all of these variations.

The embodiments described above also show that functions of various coupling mechanisms may be allocated among several components or may be combined into a single component. A locking ring, for example, may be integrally formed with the support shaft or the manipulating device, or the locking ring may be a member distinct from both.

Various modifications may be made to the specifically described embodiments without departing from the scope of the claims. For example, different kinds of locking mechanisms may be employed in addition to the particular locking mechanisms shown. The invention is not limited to locking mechanisms that squeeze components together or that seat pins in recesses. Additional locking mechanisms may include a key-and-lock mechanism, a cog mechanism, a mechanism that expands an interior component so that it engages more secure with an exterior component, or a hasp-like or clip-like fastener. The invention is not limited to any particular locking mechanism, and need not employ a locking mechanism of any type.

1. A device comprising:
a manipulating device that contacts an organ;
a support shaft;
a coupling mechanism that couples the manipulating device to the support shaft, the coupling mechanism including a first mating component and a second mating component, the first mating component and the second mating component shaped to resist separation of the manipulating device and the support shaft when the first mating component is coupled to the second mating component.

2. The device of claim 1, further comprising a third mating component, the third mating component coupling the first mating component to the second mating component.

3. The device of claim 1, wherein the support shaft comprises a vacuum tube.

4. The device of claim 1, wherein the manipulating device is formed integrally with one of the first and second mating components.

5. The device of claim 1, wherein the support shaft is formed integrally with one of the first and second mating components.

6. The device of claim 1, wherein the manipulating device is formed integrally with the first mating component and the support shaft is formed integrally with the second mating component.

7. The device of claim 1, wherein the first mating component includes at least one of a shaft member, a pin, a slot, a flange, a socket, a protrusion, a recess, a coupling ring and a screw-like thread.

8. The device of claim 1, wherein at least one of the first mating component and the second mating component includes a locking mechanism to limit the freedom of motion of the first mating component relative to the second mating component.

9. A method comprising:
engaging a manipulating device with an organ, the manipulating device being coupled to a support shaft with a coupling mechanism, the coupling mechanism having a rotationally locked configuration and rotationally unlocked configuration; and
placing the coupling mechanism in the rotationally locked configuration.

10. The method of claim 9, further comprising placing the coupling mechanism in the rotationally locked configuration by locking the coupling mechanism in the rotationally locked configuration with a locking mechanism.

11. The method of claim 9, wherein the rotationally unlocked configuration allows the manipulating device to twist relative to the support shaft.

12. The method of claim 9, further comprising applying vacuum pressure to the manipulating device to cause the manipulating device to adhere to the organ.

13. The method of claim 9, further comprising substantially supporting the weight of the organ with the manipulating device.

14. The method of claim 9, further comprising coupling the manipulating device to the support shaft with the coupling mechanism.

15. The method of claim 9, wherein engaging the manipulating device with the organ comprises engaging the manipulating device with an apex of a heart.

16. A method comprising:
coupling a manipulating device to a support shaft with a coupling mechanism;
engaging the manipulating device with an organ; and
substantially supporting the weight of the organ with the manipulating device.

17. The method of claim 16, wherein the coupling mechanism has a rotationally locked configuration and rotationally unlocked configuration.

18. The method of claim 17, further comprising placing the coupling mechanism in the rotationally locked configuration.

19. The method of claim 16, further comprising applying vacuum pressure to the manipulating device to cause the manipulating device to adhere to the organ.

20. The method of claim 16, wherein engaging the manipulating device with the organ comprises engaging the manipulating device with an apex of a heart.

21. A device comprising:
a manipulating device to manipulate an organ;
a support shaft;
a coupling ring that couples the manipulating device to the support shaft and resists separation of the manipulating device and the support shaft.

22. The device of claim 21, wherein the coupling ring includes a recess that receives a retaining ring in at least one of the manipulating device and the support shaft.

23. The device of claim 21, wherein the coupling ring includes a slot that receives a pin in at least one of the manipulating device and the support shaft.

24. The device of claim 23, wherein the slot comprises a locking slot, and wherein the locking ring is in a rotationally locked configuration when the pin is seated in the locking slot.

25. The device of claim 23, further comprising a gasket material that bears against the manipulating device and the support shaft.

26. The device of claim 23, wherein the coupling ring is integrally formed with one of the manipulating device and the support shaft.

27. The device of claim 23, wherein the support shaft includes a lumen.

28. A device comprising:
a manipulating device to manipulate an organ; and
a support shaft,
wherein one of the manipulating device and the support shaft includes a first flange, and wherein the other of the manipulating device and the support shaft includes a second flange shaped to engage with the first flange.

29. The device of claim 28, wherein the first flange defines an opening that receives the second flange.

30. The device of claim 28, further comprising a locking mechanism that when engaged places the first flange in a rotationally locked configuration relative to the second flange.

31. A device comprising:
a manipulating device to manipulate an organ; and
a support shaft,
wherein one of the manipulating device and the support shaft includes a protrusion and wherein the other of the manipulating device and the support shaft includes a complementary recess that receives the protrusion.

32. The device of claim 31, wherein the recess includes a pliable liner that deforms to allow the protrusion to be received in the recess.

33. The device of claim 31, wherein one of the manipulating device and the support shaft includes a second protrusion and wherein the other of the manipulating device and the support shaft includes a second complementary recess that receives the second protrusion.

34. The device of claim 31, further comprising a locking mechanism that when engaged places the manipulating device in a rotationally locked configuration relative to the support shaft.

35. A device comprising:
a manipulating device to manipulate an organ; and
a support shaft,
wherein one of the manipulating device and the support shaft includes a tapered member, and wherein the other of the manipulating device and the support shaft includes a socket that receives the tapered member.

36. The device of claim 35, wherein the socket includes a pliable liner that deforms to allow the protrusion to be received in the recess.

37. The device of claim 35, further comprising a locking mechanism that when engaged places the manipulating device in a rotationally locked configuration relative to the support shaft.

38. A device comprising:
a manipulating device to manipulate an organ; and
a support shaft,
wherein one of the manipulating device and the support shaft includes a flange, wherein the other of the manipulating device and the support shaft includes a flared end with an opening that receives the flange, wherein one of the flared end and the flange includes male flange, and wherein the other of the flared end and the flange includes a groove that receives the male flange.

39. The device of claim 38, wherein the male flange is one of an annular projection and a screw-like thread.

40. The device of claim 38, further comprising a grommet that engages the flared end and is shaped to increase the frictional engagement between the flange and the flared end.

41. The device of claim 40, further comprising a locking ring that engages the grommet and that further increases the frictional engagement between the flange and the flared end.
